(19) United States
(12) Patent Application Publication
(10) Pub. No.: US 2025/0375284 A1
(43) Pub. Date: Dec. 11, 2025

(54) Single Step Tissue Augmentation

(71) Applicant: Arthrex, Inc., Naples, FL (US)

(72) Inventors: Peter J. DREYFUSS, Naples, FL (US); Misty SURI, Metairie, LA (US)

(73) Assignee: Arthrex, Inc., Naples, FL (US)

(21) Appl. No.: 19/232,926

(22) Filed: Jun. 10, 2025

Related U.S. Application Data
(60) Provisional application No. 63/658,589, filed on Jun. 11, 2024.

(51) Int. Cl. A61F 2/08; A61L 27/36

(52) U.S. Cl. CPC A61F 2/0811; A61L 27/3604; A61F 2002/0829; A61F 2220/0016; A61L 2430/10

(57) ABSTRACT

Surgical constructs, assemblies, kits and methods of tissue fixation are disclosed. Soft tissue augmentation is conducted with a simple device that allows a surgeon to augment poor tissue in a most expedient and straightforward manner. The device includes an implant in the form of a molded part which has one or more barbs that are angled generally in a retrograde fashion. The implant is affixed to the graft desired to be augmented onto patient's tissue. The surgeon needs only “hook” the distal portion of the graft/implant device into patient's tissue and pull to fix the graft in contact.

### BACKGROUND

The present disclosure relates to the field of surgery and, more particularly, to constructs, systems, kits and methods for reconstructive surgeries.

### SUMMARY

Surgical constructs, assemblies, and kits are disclosed. Tissue augmentation is achieved with one or more implants provided with angled barbs which are fixed onto a graft desired to be augmented onto patient's tissue before implantation of the graft. An implant can be in the form of a molded frame provided with one or more barbs that are angled generally in a retrograde fashion. An implant can be affixed to the graft desired to be augmented onto patient's tissue. An implant and a graft can form and be provided as a unitary structure.

A surgical assembly can include an implant provided with one or more barbs and a graft used to augment poor tissue, for example, soft tissue such as rotator cuff. The implant can be fixed onto the graft before implantation of the graft to augment the soft tissue.

Methods of surgeries are also disclosed. In an embodiment, a method of tissue augmentation is conducted with a simple device that allows a surgeon to augment poor tissue in a most expedient and straightforward manner. The device includes an implant in the form of a molded part which has one or more barbs that are angled generally in a retrograde fashion. The implant is affixed to a graft desired to be augmented onto patient's tissue. The surgeon needs only “hook” the distal portion of the graft/implant assembly into patient's tissue and pull to fix the graft in contact.

### BRIEF DESCRIPTION OF THE DRAWINGS

FIG. 1 illustrates a perspective view of an exemplary implant.

FIG. 2 illustrates a top view of an exemplary assembly.

FIG. 3 illustrates a top view of another exemplary assembly.

FIG. 4 illustrates a perspective view of the assembly of FIG. 2.

FIG. 5 illustrates a perspective view of the assembly of FIG. 3.

FIG. 6 illustrates soft tissue augmentation with an exemplary assembly and at an intermediate stage of repair.

FIG. 7 illustrates a perspective top view of another exemplary assembly.

FIG. 8 illustrates a perspective top view of another exemplary assembly.

FIG. 9 illustrates another perspective view of the assembly of FIG. 7.

FIG. 10 illustrates another perspective view of the assembly of FIG. 8.

FIG. 11 illustrates soft tissue augmentation with the assembly of FIG. 8 and at an intermediate stage of repair.

### DETAILED DESCRIPTION

The present disclosure provides methods, surgical constructs, assemblies, kits and repair systems for soft tissue repairs and augmentation.

An implant can be provided with angled barbs (angled teeth) and fixed onto a graft desired to be augmented onto patient's tissue before implantation of the graft. An implant can be in the form of a molded frame provided with one or more barbs (one or more angled teeth) that can be angled generally in a retrograde fashion. Angled barbs of an implant can attach to graft. A graft can have various configurations and dimensions. A graft can be provided as a simple strip or patch having various dimensions, for example, about 2×3 mm, 10×10 mm, 12×12 mm, 10×20 mm, 20×20 mm, or 30×30 mm for larger repairs, among many others.

Soft tissue augmentation (such as rotator cuff augmentation) is typically conducted by providing additional tissue (extra tissue and/or graft) on top of the soft tissue. Augmentation is typically conducted by (i) undergoing multiple and often time difficult surgical steps such as graft unfolding and/or sequential fixation of tacks/staples that are applied separately requiring additional steps, and (ii) requiring various instrumentation and devices such as inserter, scope, graft spreader, staples and/or tacks, among others. The present disclosure provides a fast, single-step technique that can take place at the end of the soft tissue repair, without the need for using multiple steps (such as difficult unfolding of the graft over the soft tissue to be repaired, or sequential fixation of tacks/staples applied separately in multiple steps) and eliminating instrumentation such as inserters and graft spreaders. The angled barbs of the implant (with the barbs angled back) attach into the medial end of the graft and the barbs penetrate the soft tissue. By pulling on the lateral side of the graft, the barbs securely fixate within the soft tissue (as a result of the generally retrograde barb angle) and provide, therefore, secure attachment of the graft to poor soft tissue. In this manner, a surgeon can simply, easily, and securely attach soft tissue to another soft tissue, in one single step and by one hand operation, without any need to unfold the graft over the soft tissue and to use associated instrumentation, or to conduct multiple steps to apply multiple fixation devices such as staples and tacks separately and in multiple steps.

A repair system includes (i) one or more grafts for augmenting soft tissue to be repaired/improved; and (ii) one or more implants in the form of a molded frame provided with one or more barbs that are generally angled in a retrograde fashion. At least one implant can be affixed to at least one graft. An implant and a graft can be provided as a single piece, unitary structure. An implant can include any number of barbs, for example one, two, four, six, etc. barbs. A graft can include a strip or patch having dimensions of, for example, about 2×3 mm, 10×10 mm, 10×12 mm, 12×12 mm, 10×20 mm, 20×20 mm, or 30×30 mm for larger repairs. A repair system can include two strips that can be attached together. A repair system can include two strips of about 12×12 mm each that can be attached together or can be used separately for the soft tissue augmentation. Two strips of about 12×12 mm each can be employed as a 25×25 mm graft, for example. The repair system eliminates the need for multiple instrumentation as well as difficult surgical steps such as graft unfolding over soft tissue repair site. The repair system provides a simple and compact design, and strong, rapid and quick and stable fixation.

Methods of surgeries are also disclosed. An exemplary method of soft tissue repair includes inter alia the steps of: (i) attaching barbs of one or more grafts to a soft tissue to be augmented; and (ii) securing the one or more grafts to the soft tissue by fixating the barbs within the soft tissue. The method can further include the steps of pulling on a side of the graft to hook the barbs into the soft tissue; and additionally securing the side of the graft to tissue by employing a fixation device. The side of the graft that is pulled can be opposite to a side provided with the barbs. A fixation device can be a staple, tack, anchor or implant, among many others. The method can further include the steps of (i) providing an implant that includes a molded part/frame; and at least one barb or tooth extending away from a surface of the molded part/frame and in a direction about non-perpendicular to the surface of the molded part/frame; and (ii) securing the implant to one of the one or more grafts by passing the at least one barb of tooth through the graft from one surface of the graft to another, opposite surface of the graft. The tissue repair can be knotted or knotless repair. The tissue repair can be rotator cuff repair. The method can be a single-step repair. The method can be a single-handed repair.

In an embodiment, an implant can be made with one barb or tooth. In an embodiment, an implant can be made with two or more barbs or teeth. An implant can be provided with any number of barbs or teeth, for example, one, two, four, six or eight barbs (teeth). The implant can be fixed onto the graft before implantation (outside the body, preferably during manufacture so surgical staff has less work to do) to form a graft/implant assembly by passing the barbs of the implant through the graft. The implant and graft can be provided to surgeon as a single, pre-formed, pre-assembled, unitary structure (a pre-assembled graft/implant assembly). The graft can be in the form of a patch or strip cut to predefined dimensions (for example, 10×10 mm, or 20×20 mm, etc.).

The implant can be positioned and secured to the distal end of graft, such that the graft with attached implant is inserted away from the surgeon. At desired point, the surgeon pushes down while pulling the graft/implant assembly; this action causes the barbs to dig in and securely fix into the underlying tissue to be augmented. A more direct lateral/proximal traction can ideally cause more digging in of the barbs (also reducing the graft to the underlying tissue).

The tissue augmentation method eliminates the use of an inserter, graft spreader, multiple staples and tacks and similar devices and/or associated instrumentation necessary for soft tissue augmentation, providing instead a simple, economical, and rapid way of augmenting poor tissue. The tissue augmentation method is a single step tissue reinforcement that can easily and efficiently affix one soft tissue (graft) to another soft tissue, eliminating method steps and speeding up the overall surgical procedure. Barbs are fixed onto a graft before implantation of the graft to augment soft tissue (such as rotator cuff) to be repaired.

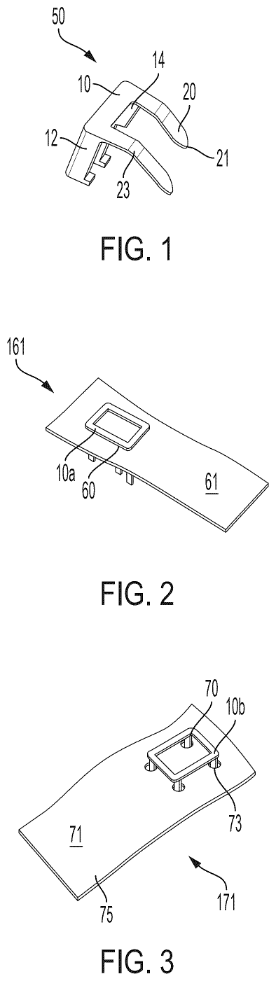

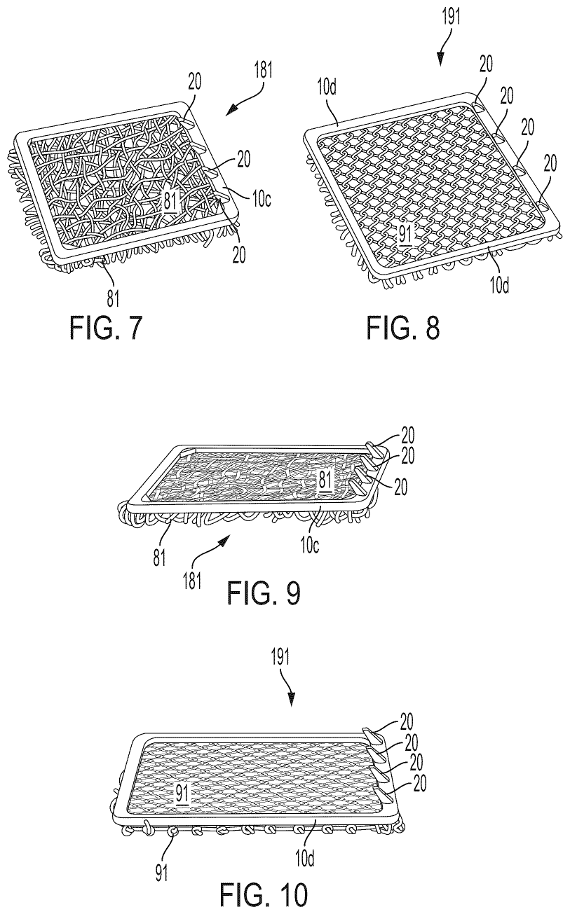

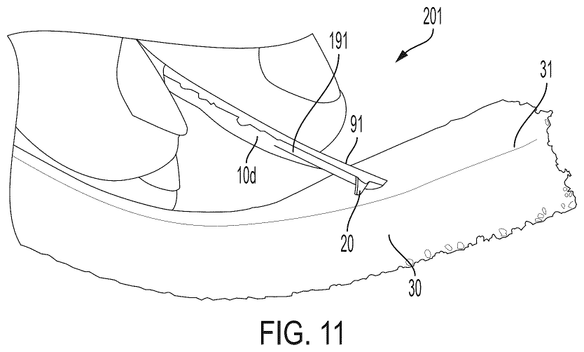

Referring now to the drawings, where like elements are designated by like reference numerals, FIGS. 1-6 illustrate exemplary implant 50, 60, 70 (molded device 50, 60, 70; barbed implant 50, 60, 70; securing device 50, 60, 70) and implant/graft assembly 161, 171 (augmenting device 161, 171; augmenting assembly 161, 171) employed for soft tissue repairs such as repair 101 (FIG. 6). FIGS. 7-11 illustrate unitary structures 181, 191 (implant/graft assembly 181, 191, augmenting device 181, 191; unitary augmenting assembly 181, 191) employed for soft tissue repairs such as repair 201 (FIG. 11). The repairs and constructs detailed below include soft tissue grafts that can be used by themselves or with additional fixation devices such as suture anchors.

FIG. 1 illustrates exemplary implant 50 in the form of a unitary structure with a molded frame 10 having a top surface 12 (first surface 12) and a bottom surface 14 (second surface 14), and a plurality of barbs 20 (teeth 20, spikes 20; protuberances 20; extensions 20; projections 20) extending away from the molded frame 10. FIG. 1 illustrates implant 50 with only two barbs; however, it must be understood that the disclosure contemplates implants with any number of barbs, depending on the configuration of the graft and of the particular soft tissue to be augmented, and as described in more detail below.

In an embodiment, frame 10 can be formed of a bioabsorbable material or a partially absorbable material, for example, a resorbing material that provides secure integration of the graft to tissue to be augmented over a desired period of time. In an embodiment, frame 10 can have a generally rectangular configuration (as shown in FIGS. 2 and 3, for example); however, frame 10 can have any other desired configuration, such as triangular, for example, depending on the desired number of barbs and/or the extent of the tissue to be repaired.

Barbs 20 of implant 50 of FIG. 1 are provided with a tip 21 that can have a generally conical configuration and a body 23 extending away from a surface of the molded frame 10, for example, from bottom surface 14. Barbs 20 can extend in a direction about non-perpendicular to the bottom surface 14, for example, at an angle different from about 90 degrees. In an embodiment, body 23 of barb 20 can be about perpendicular to surface 14 whereas tip 21 can form an angle with the body 23, for example, extends in a direction about non-perpendicular to the bottom surface 14. In an embodiment, both body 23 and tip 21 can extend in a direction about non-perpendicular to the bottom surface 14.

In one embodiment, at least one of the barbs 20 can be provided with additional fixation structures such as cuts, barbs, teeth, spikes, protuberances, or similar structures provided around at least a portion of the outer barb surface. These additional fixation devices can add to the overall fixation of the barbs 20 within soft tissue to be augmented and can also prevent backing off of the barbs from the tissue. Any additional fixation structures on barbs 20 can be provided in a same or different directions relative to the longitudinal axis of the barb 20 and can have similar and/or different sizes and configurations. In an embodiment, all barbs 20 can be provided with additional fixation structures. In an embodiment, at least one barb 20 can be provided with additional fixation structures. In yet other embodiments, the barbs can be provided without any additional fixation structures.

Barbs 20 can have a straight or curved configuration, or a combination of straight and curved. Barbs 20 are pointing backwards (angled backwards or angled retrograde) to prevent the user to push the barbs down to and through the tissue. By being angled retrograde, and provided with the specific angled configuration, the surgeon simply puts the graft with the implant over the tissue to be thickened (for example, poor rotator cuff that needs to be thickened) and, when the barb(s) are in, the surgeon simply pulls on the graft/implant assembly to secure the graft/implant assembly over the soft tissue to be thickened (augmented). As detailed below, the graft can be additionally secured to the soft tissue to be augmented on the lateral side.

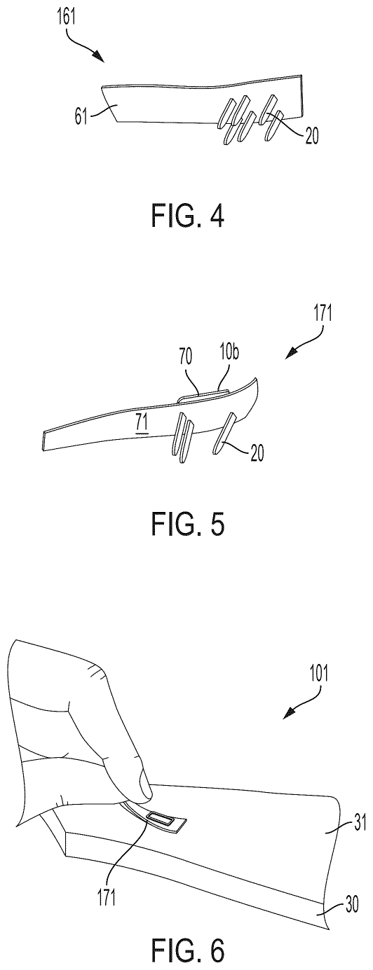

FIGS. 2-5 illustrate exemplary surgical repair systems 161, 171 (assembly 161, 171; implant/graft assembly 161, 171; implant/soft tissue graft assembly 161, 171; device 161, 171). Assembly 161 shown in FIGS. 2 and 4 includes graft 61 and implant 60 attached to it. Assembly 171 shown in FIGS. 3 and 5 includes graft 71 and implant 70 attached to it.

Graft 61, 71 can be in the form of a strip or patch having various configurations and dimensions. Graft 61, 71 can have a graft length, width and thickness that can vary depending on the size of the tissue to be augmented. In an embodiment, graft 61, 71 can have a general rectangular configuration and can be precut to desired graft dimensions (length and width) such as, for example, about 10×10 mm, 12×12 mm, 10×20 mm, 20×20 mm, or 30×30 mm for larger repairs. Graft 61, 71 can be any soft tissue graft such as synthetic graft, allograft, or autograft, or combinations thereof. Graft 61, 71 can be provided in any number and each graft can have similar or different properties (material, dimensions, etc.) from the remaining grafts. Graft 61, 71 can be in the form of precut material (precut strips) that can be further provided with starter holes (preformed holes) where the implant can attach to, for example, where the staples/barbs go through (poke through) the material graft.

Implant 60, 70 is about similar to exemplary implant 50 detailed above, in that it also includes a molded frame 10a, 10b with a top surface 12 and a bottom surface 14 with a plurality of barbs 20 (teeth 20, spikes 20; protuberances 20; extensions 20) extending away from the molded frame 10a, 10b. However, implant 60, 70 differs from implant 50 in the number of barbs 20. As such, exemplary implant 60 is provided with six barbs 20 (shown more clearly in FIG. 4). Exemplary implant 70 is provided with three barbs 20 (shown more clearly in FIG. 5). The length of the barbs of each of implant 60, 70 can differ among themselves and also among the implants. For example, the three barbs 20 of assembly 171 can have a length longer than the length of the six barbs of assembly 161. The lengths of the three barbs 20 of assembly 171 can also be all similar or different. As detailed above, barbs 20 can be further provided with additional fixation structures such as cuts, spikes, barbs etc. provided on at least an outer surface of the barbs.

In an embodiment, a repair system can include two or more strips such as grafts 61, 71 that can be attached together. For example, a repair system can include two strips of about 12×12 mm each that can be attached together or can be used separately for the soft tissue augmentation. Two strips of about 12×12 mm each can be employed as a 25×25 mm graft, for example. If two or more strips/grafts are employed, the strips can be provided in any number and each can have similar or different properties from the remaining strips/grafts. The graft dimensions are provided to allow the graft/implant to pass through an arthroscopic cannula (for example, a 12 mm PassPort Button™ cannula) for use in arthroscopic orthopedic procedures, or any other cannula. Strips provided in 12×12 mm dimensions can provide additional benefits as they can fit and be easily passed through arthroscopic portals and cannulas.

FIG. 6 illustrates a schematic soft tissue augmentation repair 101 that can be conducted with any of the implants 50, 60, 70 and assemblies 161, 171. As shown in FIG. 6, implant 70 is positioned and secured to distal end 73 of graft 71, such that assembly 171 (the graft 71 with attached implant 70) is inserted away from the surgeon and positioned on and over surface 31 of tissue 30. At desired point, the surgeon pushes down on tissue 30 to be augmented (poor soft tissue 30) while pulling the graft/implant assembly 171 at medial end 75; this action causes the barbs 20 to dig in and securely fix into the underlying tissue 30 to be augmented. A more direct lateral/proximal traction can ideally cause more digging in of the barbs 20 (also reducing the graft to the underlying tissue).

If necessary, additional fixation can be conducted to secure the remaining body of graft 71. For example, medial end 75 of graft 71 can be secured with additional fixation devices such as staples, tacks, anchors, and/or implants, among many others. If anchors are employed, the anchors can be any of suture anchors, knotted anchors, knotless anchors and/or “all suture anchors” or soft suture anchors comprising a flexible tubular sleeve or sheath and a plurality of flexible strands extending through a passage of the flexible tubular sleeve or sheath. The knotless fixation devices can be knotless anchors, for example, swivel and/or screw-in suture anchors and/or push-in anchors (such as an Arthrex SwiveLock® anchor or a PushLock® Anchor). In an exemplary embodiment, the fixation device is a knotless suture anchor such as the two-piece Arthrex PushLock® anchor, disclosed in U.S. Pat. No. 7,329,272, or an Arthrex SwiveLock® anchor, disclosed in U.S. Pat. No. 8,012,174 issued Sep. 6, 2011, and U.S. Pat. No. 9,005,246 issued Apr. 14, 2015, the disclosures of all of which are fully incorporated by reference in their entirety herein.

In an exemplary embodiment, the fixation device can be an all-suture soft anchor (soft suture anchor) provided with a soft anchor sleeve (sheath, tubular member) with two open ends and one or more flexible shuttling strands extending through the soft anchor sleeve (sheath). The at least two flexible strands may extend through the sleeve in similar or different directions and/or orientations and/or locations. The flexible sleeve with the one or more shuttling strands is secured into or onto bone (glenoid), and the strands allow passing of additional flexible strands such as tapes to pass over soft tissue and be secured into bone to approximate soft tissue to bone. Details of an exemplary soft suture anchor with a soft anchor sleeve (sheath or tubular member) and flexible shuttling strands are set forth, for example, in U.S. application Ser. No. 15/998,516 entitled “Methods of Tissue Repairs” filed on Aug. 16, 2018 (issued as U.S. Pat. No. 10,849,734 on Dec. 1, 2020), the disclosure of which is incorporated by reference in its entirety herein.

The repair 101 eliminates the need for multiple instrumentation as well as difficult surgical steps such as graft unfolding over soft tissue repair site. The repair system 161, 171 provides a simple and compact design for a strong, rapid and stable fixation.

Reference is now made to FIGS. 7-11 which illustrate additional exemplary assemblies 181 (FIGS. 7 and 9) and 191 (FIGS. 8 and 10) employed for soft tissue repairs and augmentations such as repair 201 of FIG. 11. Implant assemblies 181, 191 are about similar to assemblies 161, 171 detailed above in that they also contain grafts in the form of pre-cut strips or patches provided with molded frames containing one or more teeth or barbs; however, assemblies 161, 171 are provided to surgeons as unitary implants that are formed of materials with increased physical and biological properties which in turn confer increased ease of insertion and handling through arthroscopic portals and promote tissue grow.

As shown in FIGS. 7-10, assemblies 181, 191 are unitary structures each formed of graft/patch/mesh 81, 91 provided with incorporated fasteners therein. Grafts 81, 91 can be about similar to grafts 61, 71 described above in that they can be in the form of a strip or patch having various configurations and dimensions, and can be precut to desired graft dimensions (length and width) such as, for example, about 10×10 mm, 12×12 mm, 10×20 mm, 20×20 mm, or 30×30 mm for larger repairs. Grafts 81, 91 can be formed of one or more fibrous materials having physical properties similar to those of an air filter, with small openings (perforations, gaps, holes, etc.) having various dimensions that can provide properties such as increased elasticity, for example. Grafts 81, 91 can resemble and be in the form of embroidered patches and have properties that allow them to easily access and be brought into and through narrow arthroscopic spaces.

Grafts 81, 91 can be synthetic grafts, allografts, or autografts, or combinations thereof, or biologic materials. An exemplary-only material for graft/patch/mesh 81, 91 is ArthroFlex® bioimplant which is an acellular dermal matrix for supplemental support and covering for soft-tissue repairs; it can be used to augment reconstructions, such as rotator cuff repairs; as reinforcement for tendons and ligaments such as the Achilles; and/or to provide cushioning and support to structures, such as tuberosities. Another exemplary-only material for graft/patch/mesh 81, 91 is polyester.

Each of grafts 81, 91 is provided integral with a corresponding frame 10c, 10d having one or more teeth or barbs 20. FIGS. 7-10 illustrate frames 10c, 10d having dimensions about similar to those of the pre-cut grafts 81, 91, following the contour of the corresponding graft or patch. However, frames 10c, 10d do not need to be full length to follow the full perimeter and/or length of the grafts 81, 91. Frame 10c, 10d can be attached to mesh or graft 81, 91 by molding, gluing, bonding, snap-fitting or by any other similar process that allows secure joining and attachment of the frame to the mesh.

In an exemplary-only embodiment, and as illustrated in FIGS. 7-10, four teeth or barbs 20 are provided on only one side/edge of frame 10c, 10d. However, as detailed above, the number of teeth or barbs 20 can vary depending on the characteristics of the application. Teeth or barbs 20 can be also provided at various locations on frame 10c, 10d and not on only one side/edge. For example, in some implementations, teeth or barbs 20 can be provided on opposing barbs on both sides of the frames 10c, 10d. Some or all of the teeth can be angled retrograde (pointing backwards). In other implementations, some or all of the teeth can have an about straight configuration. The teeth or barbs are digging and/or grabbing onto soft tissue in a super easy and fast manner, with minimal input from the surgeon.

Frames 10c, 10d can also be 3D printed and can be formed of a bioabsorbable material or a partially absorbable material, for example, a resorbing material (such as resorbable plastic) or nylon that provides secure integration of the graft to tissue to be augmented over a desired period of time. In an embodiment, frame 10c, 10d can have a generally square or rectangular configuration (as shown in FIGS. 7-10, for example) that follow the shape of the graft 81, 91; however, frame 10c, 10d can have any other desired configuration, such as triangular, for example, depending on the shape of the graft, the desired number of barbs and/or the extent of the tissue to be repaired.

As in the previously described implementations, grafts 81, 91 can be provided in any number and each graft can have similar or different properties (material, dimensions, etc.) from the remaining grafts. For example, a surgeon can employ a 6 mm square implant assembly 181 in the form of a thin strip like a piece of tape that can easily fold and unfold through a small cannula (for example, an Arthrex PassPort cannula). As an alternative, a surgeon can choose to employ two 3×6 mm tapes side by side.

FIG. 11 illustrates a schematic soft tissue augmentation repair 201 with exemplary assembly 191 inserted away from the surgeon and positioned on and over surface 31 of soft tissue 30. At desired point, the surgeon pushes down on tissue 30 to be augmented (poor soft tissue 30) while pulling the graft/implant assembly 191 at the medial end; this action causes the barbs 20 to dig in and securely fix into the underlying tissue 30 to be augmented. A more direct lateral/proximal traction can ideally cause more digging in of the barbs 20 (also reducing the graft to the underlying tissue). Additional fixation can be conducted to secure the remaining body of graft or mesh 91, as detailed above with reference to assemblies 161, 171.

The implants and graft assemblies detailed above can be provided as part of a surgical kit. A surgical kit for soft tissue augmentation with tissue grafts can include: (i) one or more pre-assembled implant/graft assemblies 161, 171, 181, 191; and (ii) one or more fixation devices such as anchors. The pre-assembled implant/graft assemblies 161, 171, 181, 191 aid the surgeon in simple delivering, positioning, and fixation of the graft on top of the soft tissue to be repaired and/or augmented. The graft is fast and precisely positioned and secured to the soft tissue to be augmented and/or thickened, without the need of multiple instrumentation and multiple surgical steps. The kit can include pre-packaged (pre-assembled) implant/graft assemblies 161, 171, 181, 191. The pre-packaged implant/graft assemblies can have various dimensions and configurations (for example, implant and grafts can be tailored for specific tissue repairs, for example, rotator cuffs repairs of various sizes). The kit can include one or more inserters. An inserter can capture and hold the frame and release it fast, providing an easy way for the surgeon to easily augment tissue and allow it to grow.

A surgical kit for soft tissue augmentation with tissue grafts can include: (i) one or more implants 50, 60, 70; (ii) one or more grafts/strips/patches 61, 71, 81, 91; and (iii) one or more fixation devices such as anchors. The one or more implants 50, 60, 70 can be provided unattached to the one or more grafts/strips/patches 61, 71, 81, 91 so that attachment can be conducted intraoperatively. The surgical kit can further include (iv) one or more pre-assembled implant/graft assemblies 161, 171, 181, 191 in addition to the unassembled components.

A repair system 161, 171, 181, 191 includes (i) one or more grafts 61, 71, 81, 91 for augmenting soft tissue 30 to be repaired/improved; and (ii) one or more implants 50, 60, 70 in the form of a molded frame 10, 10c, 10d provided with one or more barbs 20 that are generally angled in a retrograde fashion. At least one implant 50, 60, 70 can be affixed to at least one graft 61, 71, 81, 91. An implant 50, 60, 70 can include any number of barbs, for example one, two, four, six, etc. barbs. A graft 61, 71, 81, 91 can include a strip 61, 71, 81, 91 or patch 61, 71, 81, 91 having dimensions of, for example, about 2×3 mm, 6×6 mm, 10×10 mm, 10×12 mm, 12×12 mm, 10×20 mm, 20×20 mm, or 30×30 mm for larger repairs. A repair system 161, 171, 181, 191 can include two strips 61, 71, 81, 91 that can be attached together. A repair system 161, 171, 181, 191 can include two strips 61, 71 of about 12×12 mm each that can be attached together or can be used separately for the soft tissue augmentation. Two strips 61, 71 of about 12×12 mm each can be employed as a 25×25 mm graft, for example. The repair system 161, 171, 181, 191 eliminates the need for multiple instrumentation as well as difficult surgical steps such as graft unfolding over soft tissue repair site. The repair system 161, 171, 181, 191 provides a simple and compact design, and strong, rapid and stable fixation.

Methods of surgeries are also disclosed. An exemplary method of tissue repair 101, 201 includes inter alia the steps of: (i) attaching barbs 20 of one or more graft assemblies 161, 171, 181, 191 to a soft tissue 30 to be augmented and (ii) securing the one or more graft assemblies 161, 171, 181, 191 to the soft tissue by fixating the barbs 20 within the soft tissue 30. The method can further include the steps of pulling on a side 25 of the graft 61, 71, 81, 91 to hook the barbs 20 into the soft tissue 30; and securing the side 25 of the graft 61, 71, 81, 91 to tissue by employing a fixation device. A fixation device can be a staple, tack, anchor (such as suture anchor) or implant, among many others. The method can further include the steps of (i) providing an implant 50, 60, 70 that includes a molded part/frame 10, 10a, 10b; and at least one barb 20 or tooth 20 extending away from a surface 14 of the molded part/frame and in a direction about non-perpendicular to the surface of the molded part/frame 10, 10a, 10b; and (ii) securing the implant 50, 60, 70 to one of the one or more grafts 61, 71 by passing the at least one barb 20 or tooth 20 through the graft 61, 71 from one surface of the graft to another, opposite surface of the graft 61, 71. The tissue repair 101, 201 can be knotted or knotless repair. The tissue repair 101, 201 can be rotator cuff repair. The repair 101, 201 can be a single-step repair. The repair 101, 201 can be a single-handed repair.

Soft tissue grafts 61, 71, 81, 91 can be any soft tissue replacement or soft tissue replacement materials. Soft tissue grafts 61, 71, 81, 91 can be allograft, autograft, xenograft or artificial graft material. In an exemplary embodiment, at least one of soft tissue grafts 61, 71, 81, 91 can be dimensioned to follow the contour and size of the surface area of the poor soft tissue to be repaired and/or augmented.

Using the repair system of the present disclosure in lieu of a typical graft that needs to be introduced to the repair site, unfolded and spread, and subsequently secured to the site with various fixation devices such as staples, graft suture anchors, etc., makes it much easier and faster for the surgeon to deliver and fixate the graft without the risk of further harm to the shoulder joint.

The soft tissue augmentation of the present disclosure enables the simple delivering, positioning, and fixation of a graft on top of the soft tissue to be repaired and/or augmented. The constructs and methods of the present disclosure allow for precise and fast placement of a graft without additional incisions and unnecessary surgical steps, as well as improved suture management at a tissue repair site.

What is claimed is:

1. A repair system for soft tissue augmentation, comprising:
a graft; and
an implant attached to the graft, wherein the implant is provided with at least one barb, and wherein the implant is secured into soft tissue to be augmented.

2. The repair system of claim 1, wherein the implant is a unitary structure with a molded frame.

3. The repair system of claim 2, wherein the implant is provided with a plurality of barbs extending away from a surface of the graft.

4. The repair system of claim 2, wherein the plurality of barbs is oriented about non-perpendicular to the molded frame.

5. The repair system of claim 2, wherein the plurality of barbs form an angle other than 90 degrees with the molded frame.

6. The repair system of claim 1, wherein the graft is a soft tissue graft.

7. The repair system of claim 1, wherein the graft has an about rectangular configuration.

8. The repair system of claim 1, wherein the graft is an allograft, an autograft, or a synthetic graft.

9. The repair system of claim 1, further comprising at least one fixation device for attaching the graft to the soft tissue to be augmented.

10. The repair system of claim 9, wherein the at least one fixation device is a staple, a tack, an implant, a knotted anchor, a knotless anchor, or an all-suture anchor.

11. The repair system of claim 9, wherein the at least one fixation device is a soft suture anchor comprising a flexible tubular sleeve or sheath and a plurality of flexible strands extending through a passage of the flexible tubular sleeve or sheath.

12. The repair system of claim 1, wherein the soft tissue to be augmented is rotator cuff.

13. A repair of a soft tissue defect, comprising:
at least one soft tissue graft attached to at least one implant and to the soft tissue defect.

14. The repair of claim 13, wherein the at least one implant includes a molded frame and one or more barbs extending away from the molded frame.

15. The repair of claim 14, wherein the molded frame is secured over a first surface of the at least one tissue graft; and wherein a second surface of the at least one tissue graft is secured over a surface of the soft tissue defect.

16. The repair of claim 14, wherein the one or more barbs extend through the at least one soft tissue graft and into the soft tissue defect.

17. The repair of claim 14, wherein the one or more barbs form an angle other than about 90 degrees with a longitudinal axis of the molded frame.

18. The repair of claim 13, wherein the at least one tissue graft is an allograft, an autograft, or a synthetic graft.

19. The repair of claim 13, wherein the at least one tissue graft is a harvested tendon.

20. The repair of claim 13, further comprising at least one fixation device.

21. The repair of claim 20, wherein the at least one fixation device is a staple, tack, anchor, or implant.

22. A surgical kit for soft tissue augmentation, comprising:
at least one soft tissue graft;
a molded implant with a plurality of teeth extending away from a surface of the molded implant, for fixation of the at least one soft tissue graft to a soft tissue to be augmented; and
a fixation device for additional fixation of the soft tissue graft to the soft tissue to be augmented.

23. The surgical kit of claim 22, wherein the at least one fixation device is a staple, a tack, an implant, or an anchor.

24. The surgical kit of claim 22, wherein the at least one soft tissue graft and the molded implant are provided as a single, unitary structure.

25. A method of soft tissue augmentation, comprising securing a soft tissue graft to a soft tissue to be augmented with an implant.

26. The method of claim 25, wherein the implant includes at least one angled barb and wherein the method further comprises:
securing the implant to a distal end of the soft tissue graft;
positioning the implant and the soft tissue graft over the soft tissue to be augmented;
pushing the implant through the soft tissue to be augmented so that the at least one angled barb penetrates the soft tissue to be augmented; and
pulling on a proximal end of the soft tissue graft to secure the at least one angled barb within the soft tissue to be augmented and to secure the soft tissue graft over the soft tissue to be augmented.

27. The method of claim 26, further comprising securing the proximal end of the soft tissue graft with an additional fixation device.

28. The method of claim 27, wherein the additional fixation device is a staple, a tack, an implant, or an anchor.

29. The method of claim 25 conducted in a single step, with a single hand, and without using a graft spreader.
